(19) United States
(12) Patent Application Publication
(10) Pub. No.: US 2004/0167112 A1
(43) Pub. Date: Aug. 26, 2004

(54) Use of estrogen receptor alpha modulators for the treatment of multiple sclerosis

(75) Inventors: M. Merle Elloso, Devon, PA (US); Robert Mitchell, Doylestown, PA (US); Douglas C. Harnish, Pennsburg, PA (US); Steven J. Adelman, Doylestown, PA (US)

Correspondence Address: WYETH, PATENT LAW GROUP, FIVE GIRALDA FARMS, MADISON, NJ, 07940, US

(73) Assignee: Wyeth, Madison, NJ

(21) Appl. No.: 10/751,543

(22) Filed: Jan. 5, 2004

Related U.S. Application Data
(60) Provisional application No. 60/438,123, filed on Jan. 6, 2003.

(51) Int. Cl. A61K 31/56; A61K 31/137

(52) U.S. Cl. 514/182; 514/649

(57) ABSTRACT

The present invention provides methods of treating an autoimmune pathology in a mammal, comprising administering an agent with estrogen receptor α agonist activity in particular a selective estrogen receptor modulator, to the mammal in an amount sufficient to decrease production of TH-1 and/or TH-2 cytokines. Also provided is a method of selecting compounds useful for the treatment of multiple sclerosis, comprising selecting a compound which has estrogen receptor α agonist activity.

### FIELD OF THE INVENTION

This invention relates generally to therapies for treating autoimmune diseases and, more specifically, to the use of compounds having estrogen receptor α (ERα) agonist activity for the treatment of autoimmune diseases. In particular, the invention relates to the use of selective estrogen receptor modulators (SERMS) for the treatment of autoimmune diseases. Furthermore, the present invention relates to methods of selecting compounds useful for the treatment of autoimmune diseases.

### BACKGROUND OF THE INVENTION

Multiple sclerosis (MS) is an autoimmune disease of the central nervous system (CNS) in which the immune system makes an inappropriate immune response to components of myelin. It is characterized by inflammation of the CNS and myelin damage. CD4⁺ T-helper-1 (TH-1) cells and their products (e.g., Tumor Necrosis Factor-α (TNF-α), Interferon-γ (IFN-γ), and metalloproteinases) mediate much of the immunopathology.

As with a number of autoimmune diseases, the incidence of multiple sclerosis is higher (2 to 3 times) in females compared to males¹. Immunomodulatory effects of estrogens in MS have been shown. For example, clinical disease is ameliorated during pregnancy, when estrogen levels are high, and worsens during the post-partum period²⁻⁴. Further, improvement in symptoms have been reported in MS patients given estradiol⁵. Estrogens appear to directly affect the function of T cells and modulation of cytokine production by T cell clones from MS patients has been shown⁶⁻⁸. In addition, inhibition of the transcription factor NF-κB κy estriol was demonstrated in these cells⁸.

Estrogens also have been shown to modulate disease activity in murine experimental autoimmune encephalomyelitis (EAE), a well-defined model for multiple sclerosis⁹⁻¹³. This model was used to test treatment with SERMS/Tissue-Selective Estrogens (TSEs) and estrogen receptor α selective agonists.

SERMS are a class of drugs which bind to the estrogen receptor and show tissue-selective effects. The SERM raloxifene, for example, has estrogen-agonistic effects on bone, lipids and clotting factors, and estrogen-antagonistic effects on the breast and uterus.¹⁹ SERMS may include 1) agents previously known as antiestrogens, such as 16-epiestriol, ethamoxytriphetol, clomiphene, and tamoxifen; 2) a 19-nortestosterone derivative, tibolone; 3) raloxifene and its analogues; and 4) newer triphenylethylene derivatives, such as droloxifene, toremifene, idoxifene, and levormeloxifene.¹⁹ SERMS compete with endrogenous estrogens for binding to the receptor and may either activate or block estrogen action.¹⁹

An object of the present invention is to provide novel methods to treat autoimmune pathologies by the administration of agents having estrogen receptor a activity, particularly SERMS.

### SUMMARY OF THE INVENTION

The present invention provides a method of treating an autoimmune pathology in a mammal, comprising administering at least one agent having estrogen receptor a agonist activity to the mammal in an amount sufficient to decrease production of TH-1 and/or TH-2 cytokines.

The present invention also provides a method of treating an autoimmune pathology in a mammal, comprising administering a selective estrogen receptor modulator to the mammal in an amount sufficient to decrease production of TH-1 and/or TH-2 cytokines.

The present invention further provides a method of selecting compounds useful for the treatment of multiple sclerosis, comprising selecting a compound which has estrogen receptor α agonist activity.

### BRIEF DESCRIPTION OF THE DRAWINGS

The invention can be more fully understood from the detailed description and the accompanying drawings that form a part of this application.

FIG. 1A shows the effect of the ER antagonist ICI on estrogen-mediated suppression of disease.

FIG. 1B shows the effect of Raloxifene vs. Compound A on EAE.

FIG. 2 shows the effect of ER-selective ligands on EAE.

FIG. 3A shows the effect of in vivo administration of ER-selective ligands on TNF-α production by splenocytes from mice with EAE.

FIG. 3B shows the effect of in vivo administration of ER-selective ligands on IL-4 production by splenocytes from mice with EAE.

FIG. 3C shows the effect of in vivo administration of ER-selective ligands on IFN-γ production by splenocytes from mice with EAE.

FIG. 3D shows the effect of in vivo administration of ER-selective ligands on IL-5 production by splenocytes from mice with EAE.

FIG. 3E shows the effect of in vivo administration of ER-selective ligands on IL-2 production by splenocytes from mice with EAE.

FIG. 3F shows the effect of in vivo administration of ER-selective ligands on IL-10 production by splenocytes from mice with EAE.

FIG. 4A shows the effect of compounds on proliferation of CD4− cells upon antigen stimulation.

FIG. 4B shows the effect of compounds on proliferation of CD4+ cells upon antigen stimulation.

FIG. 5A shows the effect of compounds on TNF-α production by effector T cells upon antigen stimulation.

FIG. 5B shows the effect of compounds on IFN-γ production by effector T cells upon antigen stimulation.

FIG. 5C shows the effect of compounds on IL-4 production by effector T cells upon antigen stimulation.

FIG. 5D shows the effect of compounds on IL-2 production by effector T cells upon antigen stimulation.

### DETAILED DESCRIPTION OF THE INVENTION

As disclosed herein, administration of an agent having estrogen receptor α agonist activity to a mammal reduces the severity of autoimmune pathologies. These effects appear to be due, in part, to the effect of such agonists on reducing the production of TH-1 and/or TH-2 cytokines by T-cells in the periphery and at the site of pathology.

Therefore, the present invention provides a method of treating an autoimmune pathology in a mammal, comprising administering an agent having estrogen receptorα agonist activity to the mammal in an amount sufficient to decrease production of TH-1 and/or TH-2 cytokines. The present invention also provides a method of treating an autoimmune pathology in a mammal, comprising administering a selective estrogen receptor modulator to the mammal in an amount sufficient to decrease production of TH-1 and/or TH-2 cytokines.

The methods of the invention can be practiced with respect to a variety of autoimmune pathologies. Such pathologies are known in the art and include but are not limited to multiple sclerosis, rheumatoid arthritis, psoriasis, autoimmune thyroiditis, uvetis, myesthenia gravis, inflammatory bowel disease and Sjögren's syndrome. In preferred embodiments of the invention, the mammal may be female, male, human or non-human.

In an embodiment of the invention, the agent having estrogen receptor α agonist activity is administered by a route selected from oral, transdermal, respiratory, subcutaneous and intravenous routes.

In preferred embodiments of the invention, the TH-1 cytokine is selected from the group consisting of TNF-α, IFN-γ and IL-2, and the TH-2 cytokine is selected from the group consisting of IL-4, IL-5 and IL-10. Those skilled in the art recognize that a TH-1 mediated immune response is characterized by secretion of pro-inflammatory cytokines, which includes TNF-α, IFN-γ, IL-2. A TH-2 mediated response is characterized by secretion of anti-inflammatory cytokines such as IL-4, IL-5 and IL-10. In one preferred embodiment of the invention, the production of TH-1 cytokines is suppressed by administration of the agent. In another preferred embodiment of the invention, the production of both TH-1 and TH-2 cytokines is suppressed. In a further embodiment of the invention, the production of TH-1 cytokines is suppressed and the production of TH-2 cytokines is increased.

As a preferred embodiment, the ERα agonist exhibits an anti-inflammatory activity, e.g. a reduction in NF-κB activity. In another preferred embodiment, the ERα agonist is non-steroidal.

In a further embodiment of the invention, the SERM is selected from the group comprising raloxifene, tamoxifen, lasofoxifene, idoxifene, droloxifene, bazedoxifene, toremifene and their derivatives and analogs. In another embodiment of the invention the selective estrogen receptor modulator exerts a biological effect on the brain or central nervous system.

The present invention also provides a method of selecting compounds useful for the treatment of multiple sclerosis, comprising selecting a compound which has estrogen receptor α agonist activity. Conventional assays for assaying in vitro agonist activity, using receptors such as luciferase, are well known in the art. Illustrative of agonist assays are the following publications which are incorporated by reference for their ERα agonist assays: Lyttle CR, Damian-Matsumura P., Juul H., Bult T R, Human estrogen receptor regulation in a yeast model system and studies on receptor agonists and antagonists, J. Steroid Biochem Mol Biol 42:677-685 (1992); Katzenellenbogen B S, Bhardwaj B, Fang H, Ince B A, Pakdel F, Reese J C, Schodin D, Wrenn C K, Hormone binding and transcription activation by estrogen receptors: analyses using mammalian and yeast systems, J Steroid Biochem Mol Biol 47:39-48 (1993); PCT International Publication No. WO 00/37681; Webb P, Lopez G N, Greene G L, Baxter J D, Kushner P J, 1992, The limits of the cellular capacity to mediate an estrogen response, Mol Endocrinology, 6(2):157-67. Preferably, in such assays, an “estrogen receptor αagonist” is defined as a compound that substantially mimics ER-α activity of 17-β estradiol as measured in the selected assay for estrogenic activity.

In a preferred embodiment of the invention, the compound is a SERM. In a further embodiment of the invention, the compound decreases TNFα production by at least about 20%-100%, as described in Example II herein. In alternative embodiments, the decrease may be at least 30, 40, 50, 60 or 80%.

Definitions of Abbreviations and Terms:

The following definitions are provided for the full understanding of terms and abbreviations used in this specification.

As used herein and in the appended claims, the singular forms “a”, “an” and “the” include the plural reference unless the context clearly indicates otherwise. Thus, for example, a reference to “an estrogen receptor α agonist” includes a plurality of such agonists.

The abbreviations in the specification correspond to units of measure, techniques, properties or compounds as follows: “μg” means microgram(s), “ml” means milliliter(s), “μM” means micromole(s), “mM” means millimole(s), “s.c.” means subcutaneous, “i.p.” means intraperitoneal, and “p.o” means per oral.

“Multiple sclerosis” is abbreviated MS.

“Central nervous system” is abbreviated CNS.

“T-helper-1” and “T-helper-2” are abbreviated TH-1 and TH-2, respectively.

“Tumor Necrosis Factor-α” is abbreviated TNF-α.

“Interferon-γ” is abbreviated IFN-γ.

“Nuclear Factor-κB” is abbreviated NF-κB.

“Experimental Autoimmune Encephalomyelitis” is abbreviated EAE.

“Selective Estrogen Receptor Modulators” is abbreviated SERMS.

“Tissue Selective Estrogens” is abbreviated TSEs.

“Estrogen receptor” is abbreviated ER.

“Interleukin” is abbreviated IL.

“Proteolipid protein peptide” is abbreviated PLP.

“Complete Freund's adjuvant” is abbreviated CFA.

“Post transfer” is abbreviated PT.

As used herein, the term “autoimmune pathology” refers to a pathology mediated by a detrimental autoimmune response. In most autoimmune pathologies, T cells recognize a host component in one or more tissues as foreign and attack that tissue.

The term “treatment” as used herein includes preventative (e.g. prophylactic), curative, or palliative treatment and “treating” as used herein also includes preventative, curative and palliative treatment. “Treating”, with reference to autoimmune pathology, refers to any observable effect of the treatment. The beneficial effect can be evidenced by delayed onset of clinical symptoms in a susceptible mammal, a reduction in severity of some or all clinical symptoms of the disease, a slower progression of the disease, a reduction in the number of relapses of the disease, a reduction in the number or activity (e.g. cytokine secretion) of pathogenic T cells at the site of pathology or in the circulation, an improvement in the overall health or well-being of the individual, or by other parameters well known in the art that are specific to the particular disease.

As disclosed herein, the term “agent having estrogen receptor α activity” is an agent that exhibits ERα activity and includes but is not limited to selective estrogen receptor modulators and tissue-selective estrogens. The term may also include partial agonists, peptides, polypeptides, genes, gene fragments, non-peptide small molecules, natural products, antisense DNA and mRNA.

As used herein, the term “mammal” refers to a human, a non-human primate, canine, feline, bovine, ovine, porcine, murine or other veterinary or laboratory mammal. Those skilled in the art recognize that a therapy which reduces the severity of an immune pathology in one species of mammal is predictive of the effect of the therapy on another species of mammal. The skilled person also appreciates that credible animal models of human immune pathologies are known, including EAE, which is a credible animal model of multiple sclerosis.

An “amount effective to decrease production of TH-1 and/or TH-2 cytokines” refers to an amount effective, at dosages and for periods of time necessary, to achieve the desired result of treating autoimmune pathology. It will be appreciated that the amount of estrogen receptor α agonist effective to decrease production of TH-1 and/or TH-2 cytokines in the methods of the present invention will vary from individual to individual not only with the particular agonist selected, the route of administration, and the ability of the agonist to elicit a desired response in the individual, but also with factors such as the disease state or severity of the condition to be alleviated, age, sex, weight of the individual, the state of being of the patient, and the severity of the pathological condition being treated, concurrent medication or special diets then being followed by the particular individual, and other factors which those skilled in the art will recognize, with the appropriate dosage ultimately being at the discretion of the attendant physician. Dosage regimens may be adjusted to provide the improved therapeutic response. An “amount effective to decrease production of TH-1 and/or TH-2 cytokines” is also one in which any toxic or detrimental effects of the agonist is outweighed by the therapeutically beneficial effects.

Preferably, the estrogen receptor α agonists are administered in the methods of the present invention at a dosage and for a time such that the production of TH-1 and/or TH-2 cytokines is decreased as compared to production of these cytokines at the start of treatment. Such treatment can also be beneficial to reduce the overall severity of symptoms of autoimmune disease, as compared to the severity of symptoms prior to the start of the treatment. In a preferred embodiment, dosages range from 0.5 mg/kg/day to 500 mg/kg/day, and, alternatively, at least about 10, 50, 100 or 150 mg/kg/day.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Effect of In Vivo Administration of Compounds in Animal Model of Multiple Sclerosis

This Example shows that, in an animal model of multiple sclerosis, in vivo administration of estrogen receptor α selective agonists results in delayed onset and decreased incidence and severity of disease.

Materials and Methods

Animals. Twenty-five intact female (6-8 weeks old) SJL mice (Jackson Laboratories, Bar Harbor, Me.) were used as donor mice in the adoptive transfer model of EAE.

Induction of Experimental Autoimmune Encephalomyelitis (EAE). EAE was induced by the adoptive transfer of PLP sensitized spleen cells using a modification of methods previously described.²⁰ Mice were immunized with proteolipid protein peptide 139-151 (PLP) emulsified in complete Freund's adjuvant (CFA). Each animal received 150 μg of PLP in a volume of 0.2 ml CFA that contained 4 mg/ml heat killed and dried Mycobacterium tuberculosis (H37RA strain). The PLP/CFA emulsion was injected s.c. in two sites (on the back, and at the base of the tail). 0.1 ml was injected at each site. Ten days later, the mice were euthanized and the spleens were collected. Single cell suspensions were made from the spleens. After lysis of red blood cells, the cells were cultured at a concentration of 5×10⁶ cells/ml for 3 days in 75 cm² tissue culture flasks in RPMI-10 (RPMI medium containing 10% heat-inactivated fetal bovine serum, 100 U/ml penicillin, 100 μg/ml streptomycin, 2 mM glutamine, 50 μM 2-mercaptoethanol). PLP was added at a final concentration of 5 μg/ml. The cells were incubated at 37° C. in 5% CO₂. After the incubation, PLP-stimulated effector cells were harvested, washed with phosphate-buffered saline and injected i.p. into ovariectomized female (6-8 weeks old) SJL mice (1.5×10⁷ cells/mouse). Onset of disease typically occurs 7-14 days post-transfer (PT) of cells.

The degree of disease severity was monitored daily using the scale shown in

TABLE 1
Scale of Disease Severity

| | |
|---|---|
| 0 | no overt signs of disease |
| 1 | limp tail |
| 2 | limp tail/hind limb weakness (observed as a waddling gait; |
| | mouse's hind limbs fall through wire cage tops) |
| 3 | partial hind limb paralysis (mouse can no longer maintain |
| | rump posture but can still move one or |
| | both limbs to some extent) |
| 4 | complete hind limb paralysis (complete loss of |
| | movement in hind limbs; animal |
| | drags hind limbs; can grasp bar and pull |
| | itself up with no difficulty) |
| 5 | complete hind limb paralysis and/or mild fore |
| | limb weakness (can still grasp bar; |
| | but has some difficulty pulling itself up) |
| 6 | complete hind limb paralysis with severe |
| | fore limb weakness or paralysis; |
| | moribund |

To evaluate the effect of compounds on disease, recipient mice were administered compounds daily (s.c. or p.o.) at the doses indicated, using a 10% ethanol/90% corn oil vehicle. Control animals received vehicle only. Mice were dosed beginning 5-7 days prior to the adoptive transfer of donor cells.

Histological Analysis. At peak disease (14 days PT), mice were euthanized with CO₂. Brains and spinal cords were removed at necropsy and fixed in 10% buffered formalin. Brains were cut into three segments (roughly, anterior cerebrum, midbrain and cerebellum) and embedded as a single block. The spinal cord was decalcified in 10% HCl and cut into cervical, thoracic and lumber segments embedded as a single block. A standard H&E (hematoxylin and eosin) glass slide was prepared from each tissue block (brain and spinal cord) from each mouse submitted with two resulting H&E slides per mouse evaluated.

Slides were evaluated and lesions seen were graded subjectively for presence (P=present) and/or severity. Severity grades are O=WNL (within normal limits), 1=slight or minimal, 2=mild, 3=moderate, 4=marked, and 5=severe. The location of findings, relative to the organ, was denoted as perivascular, periventricular, ependymal or meningeal, and also as focal (in small areas or not throughout the section), or diffuse (throughout the section examined). Focal was defined as very localized and not affecting every structure. Findings not defined as focal were diffuse or affecting every structure (e.g. all vessels). Leukocytes seen were primarily lymphocytes and macrophages with occasional neutrophils. Demyelination was observed as distinct open holes in white matter tracts in the spinal cord.

Typical murine models of EAE have scattered foci of slight to moderate-sized aggregates of leukocytes and rarely have diffuse infiltrates in the affected tissues.

Analysis of PLP-specific Recall Responses: Cytokine Production. To examine the effect of compounds administered in vivo on cytokine production by splenocytes from mice with EAE, mice were euthanized with CO₂ at peak disease (14 days PT), and spleens were collected. Spleens were individually processed into single cell suspensions. After lysis of red blood cells, the cells were resuspended in RPMI-10 and were cultured in 24-well tissue culture plates at a concentration of 5×10⁶ cells/ml. Cells were stimulated with 5 μg/ml PLP. Supernatants were collected after 3 days and frozen until use at −20° C. Cytokines (TNF-α, IFN-γ, IL-5, IL-4, IL-2) were measured in the supernatants using a commercially available flow cytometry kit (Cytometric Bead Array, Becton Dickinson BioSciences, San Diego, Calif.). IL-10 was measured using an IL-10-specific ELISA kit (Becton Dickinson BioSciences).

To examine in vitro the effect of compounds on cytokine production by PLP-primed effector cells, SJL mice were immunized with PLP emulsified in CFA. After 10 days, spleens were collected and single cell suspensions were made. After lysis of red blood cells, splenocytes were stimulated with 5 μg/ml PLP in the presence of compound for 3 days at 37° C., 5% CO₂. Control samples were not stimulated and cultured in medium only (“medium”). Compounds were added at a final concentration of 1 μM. After the 3-day incubation, supernatants were collected and stored at −20° C. Cytokines (TNF-α, IFN-γ, IL-5, IL-4, IL-2) were measured in the supernatants using a Cytometric Bead Array kit.

Effect of compounds on proliferation of effector T cells upon antigen stimulation To examine the effect of proliferation of effector T cells in vitro in response to PLP stimulation, T-cell proliferation was examined by flow cytometry using the following assay. SJL mice were immunized with PLP emulsified in CFA. After 10 days, spleens were collected and single cell suspensions were made. After lysis of red blood cells, splenocytes were labeled with carboxy fluorescein succinimidyl ester (CFSE). CFSE-labeled cells were then incubated with PLP for 3 days at 37° C., 5% CO₂. Compounds were added at a final concentration of 1 μM. To determine the percentage of CD4⁺ cells that divided, the CFSE-labeled cells were stained with antibodies specific for the CD4 marker prior to flow cytometric analysis.

Results

I. Effect of SERMs/Tissue-Selective Estrogens on EAE induced by the Adoptive Transfer of PLP-primed Effector Cells

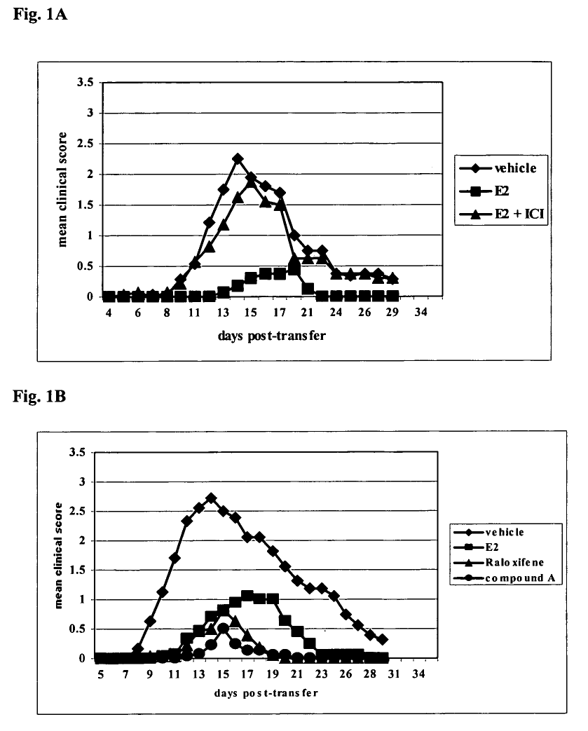

As shown previously, treatment with 17β-estradiol (E2) resulted in a delay in onset as well as decreased incidence and severity of disease (FIG. 1A; refs. 9, 11-13). To determine whether the protective effects of estrogen in this model 10 were estrogen receptor-mediated, mice were treated with both E2 and the estrogen receptor antagonist ICI 182,780. ICI abolished the effect of E2 on disease (FIG. 1A).

Treatment with E2 or with the SERMs raloxifene or Compound A [2-(hydroxyphenyl)-3-methyl-1-[4-(2-piperidin-1-yl-ethoxy)benzyl]-1H-indol-5-ol hydrochloride monohydrate] resulted in a delay in onset as well as decreased incidence and severity of disease (FIG. 1B and Table II). Consistent with the effects of these compounds on the clinical signs of disease, there was a reduction in the amount of inflammatory cells infiltrating the spinal cords and brains from mice treated with Compound A compared to mice from other treatment groups. In addition, no demyelination was detected in spinal cords from Compound A-treated mice.

TABLE II
Histological Findings

| | | | Spinal Cord | |
|---|---|---|---|---|
| | | | Meningeal/ | |
| | Brain Meningeal/ | | Perivascular | |
| | Perivascular | Distribution | Leukocyte | Spinal Cord |
| Treatment | Leukocyte Infiltrates | of Brain Lesions | Infiltrates | Demyelination |
| Vehicle | 3/3^{a} (2.33)^{b} | Meningeal, Perivascular | 3/3 (2) | 3/3 (2) |
| | | and Periependymal | | |
| E2 | 4/4 (2.5) | Meningeal, Perivascular | ¾ (1.7) | ¾ (1.3) |
| | | and Periependymal | | |
| Raloxifene | 5/5 (2.4) | Meningeal, Perivascular | 5/5 (1.4) | 5/5 (1.2) |
| | | and Periependymal | | |
| Compound A | 3/5 (2.7) | Meningeal, Perivascular | 1/5 (1) | 0/5 |
| | | and Periependymal | | |

^{a}= number of brains with finding/total number evaluated
^{b}= average severity grade of lesions seen;
0 = within normal limits,
1 = slight,
2 = mild,
3 = moderate,
4 = marked,
5 = severe

II. Effect of Estrogen Receptor-Selective Agonists on EAE induced by the Adoptive Transfer of PLP-Primed Effector Cells

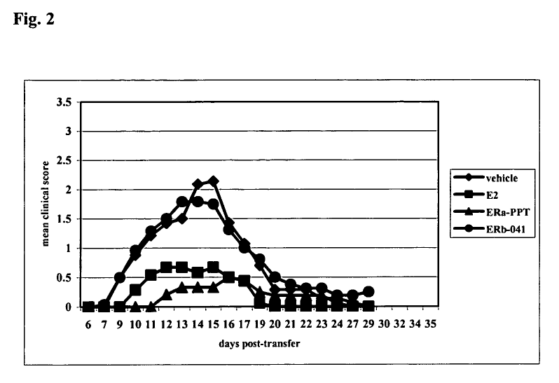

Treatment with E2 or the ERα-selective agonist PPT (propylpyrazole triol) resulted in a delay in onset as well as decreased incidence and severity of disease (FIG. 2). In addition, mice treated with PPT had reduced inflammation in the brain and spinal cords compared with mice treated vehicle, or with an ERβ-selective agonist (Table II). Histologic examination revealed that mice which were administered PPT had the most normal tissues compared to the vehicle control mice. All four had slight leukocyte infiltrates in the meninges but only at the base of the brain (around the hindbrain/cerebellum/pons/medulla on the ventral surfaces only). None of these mice had spinal cord lesions; the spinal cords of these mice were all within normal limits.

TABLE III

| | | | Spinal Cord | |
|---|---|---|---|---|
| | | | Meningeal/ | |
| | Brain Meningeal/ | | Perivascular | |
| | Perivascular Leukocyte | Distribution | Leukocyte | Spinal Cord |
| Treatment | Infiltrates | of Brain Lesions | Infiltrates | Demyelination |
| Vehicle | 4/4^{a} (2.75)^{b} | Periventricular and | 4/4 (2) | 1/4 (1) |
| | | Ependymal | | |
| E2 | 3/4 (1.7) | Meningeal | 2/4 (1) | 1/4 (1) |
| ERα-PPT (ERα) | 4/4 (1) | Base of the Brain | 0/4 | 0/4 |
| ERβ-041 (Erβ) | 4/4 (2.3) | Periventricular and | 2/4 (1.5) | 2/4 (1.5) |
| | | Ependymal | | |

^{a}= number of brains with finding/total number evaluated
^{b}= average severity grade of lesions seen;
0 = within normal limits,
1 = slight,
2 = mild,
3 = moderate,
4 = marked,
5 = severe

III. Effect of ER-Selective Agonists on PLP-specific Recall Responses: Cytokine Production

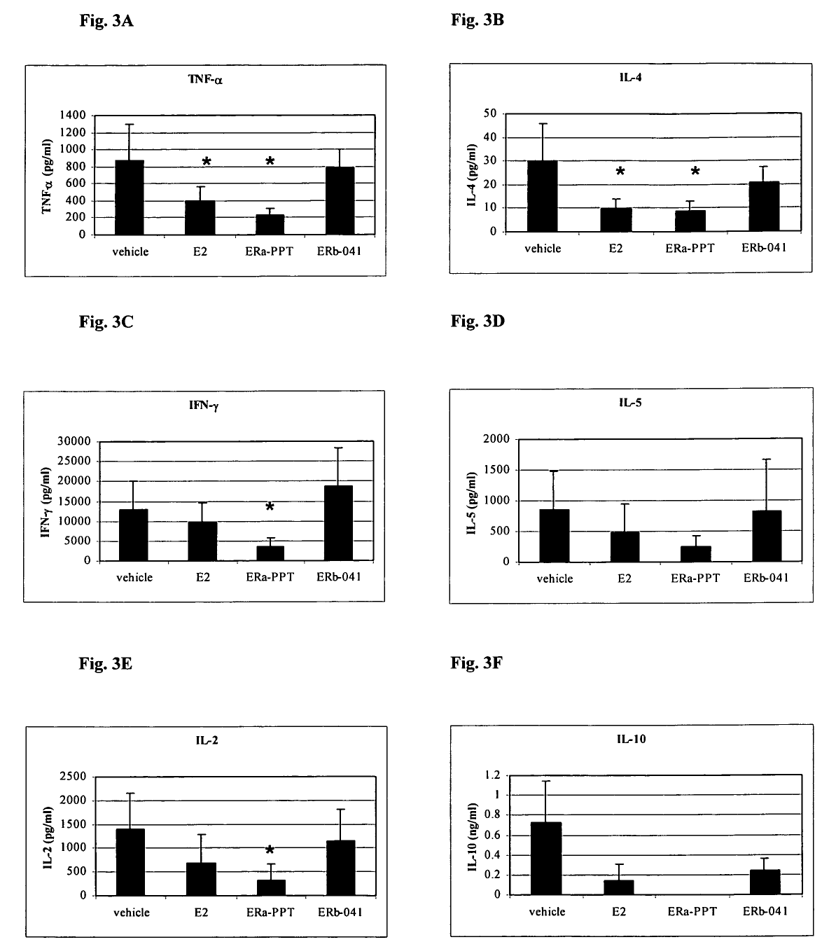

Consistent with the effect of the ERα-selective agonist PPT on disease, treatment of mice with PPT resulted in decreased cytokine production upon stimulation of splenocytes with PLP in vitro (FIG. 3). Both Th1/pro-(TNF-α, IFN-γ, IL-2) and Th2/anti-inflammatory (IL-4, IL-5, IL-10) cytokines were suppressed by in vivo treatment with PPT, indicating that PPT may suppress disease by inhibiting T cell activation, rather than by immune deviation from a pathogenic Th1 response to a protective Th2 response. In contrast, the ERα-selective agonist had no effect on cytokine production (*, p<0.05 compared to vehicle group).

### Example II

### Effect of Compounds on Antigen-Specific Immune Responses In Vitro

The effect of tissue selective estrogens (Compound A) and ERα-selective ligands (PPT) on antigen-specific immune responses was examined in vitro.

A. Effect of Compounds on Proliferation of Effector T Cells upon Antigen Stimulation

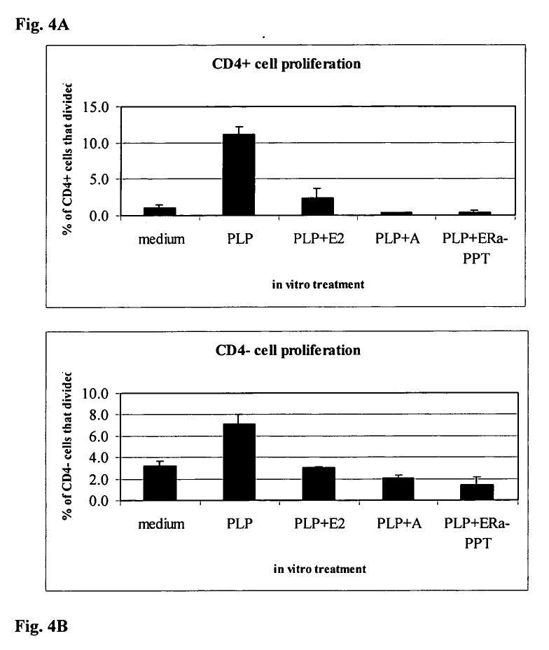

Treatment of PLP-primed effector cells with the tissue-selective estrogen Compound A or with ERα-selective agonist PPT resulted in decreased proliferation of antigen-specific T cells (FIG. 4). Proliferation of both CD4+ and CD4− cell populations was suppressed. These results suggest that each of these compounds may potentially act in part by limiting the clonal expansion of antigen-specific T cells.

B. Effect of Compounds on Cytokine Production by Effector Cells upon Antigen

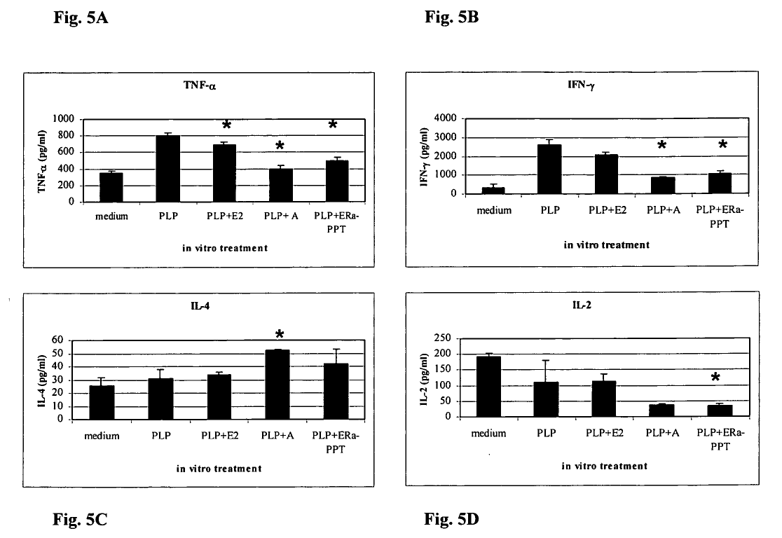

Treatment of PLP-primed effector cells with the tissue-selective estrogen Compound A or with α-selective agonist (PPT) resulted in decreased cytokine production upon antigen stimulation (FIG. 5). Both compounds inhibited the production of the pro-inflammatory (TH-1) cytokine TNF-α. Both Compound A and the ERα-selective agonists PPT also suppressed IFN-γ production. Incubation of these cells with the tissue-selective estrogen Compound A also resulted in a concomitant increase in the anti-inflammatory cytokine IL-4, whereas the other compounds had no effect. These results suggest that ERα-selective agonists (PPT) may have different effects than tissue-selective estrogens on antigen-specific cytokine production. Whereas the former may suppress EAE by inhibiting the production of pro-inflammatory (TH-1) cytokines, tissue-selective estrogens may in addition promote immune deviation to a protective anti-inflammatory/TH-2 immune response.

### Conclusions

Treatment with the SERMs/TSEs raloxifene and Compound A, as well as the ERα-selective agonist PPT, suppressed EAE, resulting in delayed onset of disease, as well as decreased incidence and severity. The suppression of the clinical signs of disease in mice treated with these compounds was associated with reduced pathology and leukocyte infiltration in the brains and spinal cords. This suggests that these compounds may reduce disease by limiting trafficking of pathogenic cells into the brains and spinal cords, for example, by decreasing adhesion molecule expression and/or by affecting chemokine/chemokine receptor expression. Suppression of disease induced by the adoptive transfer of PLP-primed effector cells with SERMs/TSEs and ERα-selective agonists suggests that these compounds have the capacity to alter the activity of encephalitogenic effector cells. These findings are in contrast to the notion that differentiated effector cells are more refractory to the effects of estrogens compared with naive cells¹³.

In vitro data presented herein suggests that ERα-selective agonists, preferentially SERMS or ER-anti-inflammatory ligands, may have direct effects on antigen-specific T cell proliferation and cytokine production, thereby limiting the expansion and differentiation of pathogenic T cells. All three types of ligands are effective in suppressing the production of the pro-inflammatory (TH-1) cytokines. Tissue-selective estrogens, however, may in addition promote the production of protective anti-inflammatory (TH-2) cytokines, which suggests that these molecules may have differential effects on PLP-specific immune responses.

The observation that SERMs/TSEs are capable of altering the course of disease in this model is somewhat surprising, given the known effects of SERMs and estrogen antagonists in murine systemic lupus erythematosus, the mouse model for lupus. SERMs have been shown to have beneficial therapeutic effects in lupus, an autoimmune disease in which the disease is exacerbated by estrogens⁴¹⁻¹³. Since SERMs appear to act in an antagonist fashion in lupus, it was anticipated that SERMs would have similar antagonist activity in EAE. Therefore, the prediction would be that SERMs would either have no effect, or would exacerbate EAE. Instead, SERMs demonstrated disease-suppressing activity.

### References

1. Whitacre C. C. 2002. Sex differences in autoimmune disease. Nat Immunol. 2:777-780.

2. Abramsky O. 1994. Pregnancy and multiple sclerosis. Ann Neruol. 36(suppl):S38-S41.

3. Confavreux C, Hutchinson M, Hours M. M. et al. 1998. Rate of preganacy-related relapse in mupltiple sclerosis. Pregnancy in Multiple Sclerosis Group. N Engl J Med. 339:285-291.

4. Damek D. M., Shuster E. A. 1997. Pregnancy and multiple sclerosis. Mayo Clin Proc. 72:977-989.

5. Sicotte N. L, Liva S. M., Klutch R. et al. 2002. Treatment of multiple sclerosis with the pregnancy hormone estriol. Ann Neurol. 52:421-428.

6. Gilmore W., Weiner L. P., Correale J. 1997. Effect of estradiol on cytokine secretion by proteolipid protein-specific T cell clones isolated from multiple sclerosis patients and normal control subjects. J. Immunol. 158:446-451.

7. Correale J. Arias M., Gilmore W. 1998. Steroid hormone regulation of cytokine secretion by proteolipid protein-specific CD4+ T cell clones isolated from multiple sclerosis patients and normal control subjects. J. Immunol. 161:3365-3374.

8. Zang Y. C. Q. Halder J. B., Hong, J. Rivera V. M., Zhang J. Z. 2002. Regulatory effects of estriol on T cell migration and cytokine profile: inhibition of transcription factor NF-κB. J. Neuroimmunol. 124:106-114.

9. Jansson L., Olsson T., Holmdahl R. 1994. Estrogen induces a potent suppression of experimental autoimmune encephalomyelitis and collagen-induced arthritis in mice. J Neuroimmunol. 53:203-207.

10. Kim S., Liva S. M., Dalal M. A., Verity M. A., Voskuhl R. R. 1999. Estriol ameliorates autoimmune demylinating disease. Implications for multiple sclerosis. Neurol. 52:1230-1238.

11. Offner H., Adlard K., Zamora A., Vandenbark A. A. 2000. Estrogen potentiates treatment with T-cell receptor protein of female mice with experimental encephalomyelitis. J Clin Invest. 105:1465-1472.

12. Ito A., Bebo B. F., Jr., Matejuk A., Zamora A., Silverman M., Fyfe-Johnson A., Offner H. 2001. Estrogen treatment down-regulates TNF-α production and reduces the severity of experimental autoimmune encephalomyelitis in cytokine knockout mice. J Immunol. 167:542-552.

13. Bebo B. F., Jr., Fyfe-Johnson A., Adlard K., Beam A. G., Vandenbark A. A, Offner H. 2001. Low-dose estrogen therapy ameliorates experimental autoimmune ensephalomyelitis in two different inbred mouse strains. J Immunol. 166:2080-2089.

14. Duvic M., Steinberg A. D., Klassen L. W. 1978. Arthritis and Rheumatism. 21:414417.

15. Sturgess A. D., Evans D. T. P., Mackay I. R., Riglar A. 1984. Effects of the oestrogen antagonist tamoxifen on disease indices in sytemic lupus erythematosus. J. Clin. Lab. Immunol. 13:11-14.

16. Sthoeger Z. M. Bentwich Z., Zinger H., Mozes E. 1994. The beneficial effect of the estrogen antagonist, Tamoxifen, on experimental lupus erythematosus. J. Rheumatol. 21:2231-2238.

17. Apelgren L. D., Bailey D. L., Fouts R. L., Short L., Bryan N., Evans G. F., Sandusky G. E., Zuckerman S. H., Glasebrook A., Bumol T. F. 1996. The effect of a selective estrogen receptor modulator on the progression of spontaneous autoimmune disease in MRL Ipr/Ipr mice. Cell. Immunol. 173:55-63.

18. Wu W-M., Lin B-F., Su Y-C., Suen J-L., Chiang B-L. 2000. Tamoxifen decreases renal inflammation and alleviates disease severity in autoimmune NZB/W F1 mice. Scand. J. Immunol. 52:393-400.

19. Kankaanpaa J T J. October, 1999. Raloxifene—a muiltifunctional medicine: review. APIMALL General Practice Issue 1.0.

20. Leonard, J. P., Waldburger K. E., Goldman S. J., 1995. J. Exp. Med. 181 :P381-386.

What is claimed is:

1. A method of treating an autoimmune pathology in a mammal, comprising administering at least one agent having estrogen receptor α agonist activity to the mammal in an amount sufficient to decrease production of TH-1 and/or TH-2 cytokines.

2. The method of claim 1, wherein the autoimmune pathology is selected from the group consisting of multiple sclerosis, rheumatoid arthritis, psoriasis, autoimmune thyroiditis, uvetis, inflammatory bowel disease and Sjögren's syndrome.

3. The method of claim 1, wherein the mammal is female.

4. The method of claim 1, wherein the mammal is male.

5. The method of claim 1, wherein the mammal is human.

6. The method of claim 1, wherein the mammal is non-human.

7. The method of claim 1, wherein the agent is administered by a route selected from oral, transdermal, respiratory, subcutaneous and intravenous routes.

8. The method of claim 1, wherein the TH-1 cytokine is selected from the group consisting of TNF-α, IFN-γ and IL-2.

9. The method of claim 1, wherein the TH-2 cytokine is selected from the group consisting of IL-4, IL-5 and IL-10.

10. The method of claim 1, wherein the agent decreases Nuclear Factor-κB activity.

11. The method of claim 1, wherein the agent is non-steroidal.

12. The method of claim 1 wherein the agent is a selective estrogen receptor modulator administered in an amount sufficient to decrease production of TH-1 and TH-2 cytokines.

13. The method of claim 12, wherein the autoimmune pathology is selected from the group consisting of multiple sclerosis, rheumatoid arthritis, psoriasis, autoimmune thyroiditis, uvetis, inflammatory bowel disease and Sjögren's syndrome.

14. The method of claim 12, wherein the mammal is female.

15. The method of claim 12, wherein the mammal is male.

16. The method of claim 12, wherein the mammal is human.

17. The method of claim 12, wherein the mammal is non-human.

18. The method of claim 12, wherein the selective estrogen receptor modulator is administered by a route selected from oral, transdermal, respiratory, subcutaneous and intravenous routes.

19. The method of claim 12, wherein the TH-1 cytokine is selected from the group consisting of TNF-α, IFN-γ and IL-2.

20. The method of claim 12, wherein the TH-2 cytokine is selected from the group consisting of IL-4, IL-5 and IL-10.

21. The method of claim 12, wherein the selective estrogen receptor modulator decreases Nuclear Factor-κB activity.

22. The method of claim 12, wherein the selective estrogen receptor modulator is selected from the group consisting of raloxifene, tamoxifen, lasofoxifene, idoxifene, droloxifene, bazedoxifene, and toremifene.

23. A method of selecting compounds useful for the treatment of multiple sclerosis, comprising selecting a compound which has estrogen receptor α agonist activity.

24. The method of claim 23, wherein the compound is a selective estrogen receptor modulator.

25. The method of claim 23, wherein the compound decreases TNFα production by at least about 20%.
